(19) United States
(12) Patent Application Publication
(10) Pub. No.: US 2025/0012004 A1
(43) Pub. Date: Jan. 9, 2025

(54) CLOTH STRUCTURE HAVING ELECTRIC HEATING AND INSULATION AND ELECTRIC STIMULATION COATINGS AND PREPARATION METHOD THEREFOR

(71) Applicant: XUZHOU H-GURAD BIOTECHNOLOGY CO., LTD, Xuzhou, Jiangsu (CN)

(72) Inventors: Wen Hsien SUN, Fuzhou, Fujian (CN); Yu QIU, Fuzhou, Fujian (CN); Hao XIONG, Fuzhou, Fujian (CN); Ping LI, Fuzhou, Fujian (CN); Zhenjing KANG, Fuzhou, Fujian (CN)

(73) Assignee: XUZHOU H-GURAD BIOTECHNOLOGY CO., LTD, Xuzhou, Jiangsu (CN)

(21) Appl. No.: 18/709,510

(22) PCT Filed: Nov. 11, 2022
(86) PCT No.: PCT/CN2022/131299

§ 371 Date: May 13, 2024

(30) Foreign Application Priority Data
Nov. 12, 2021 (CN) 202111339652.4

(51) Int. Cl. D06M 10/06; A61F 7/00; H05B 3/12; H05B 3/34

(52) U.S. Cl. CPC D06M 10/06; H05B 3/12; H05B 3/347; A61F 7/007; D10B 2401/18; D10B 2403/02431; H05B 2203/017

(57) ABSTRACT

A cloth structure having electric heating and electric stimulation coatings and a preparation method therefor are provided. The cloth structure includes a base cloth, an electric heating coating, an insulating layer and an electric stimulation coating, the base cloth has a first surface; the electric heating coating is attached to the first surface; the electric stimulation coating is attached to the electric heating coating via the insulating layer, the electric heating coating and the electric stimulation coating are separately electrically connected to a control system; the control system controls the electric heating coating to generate heat, and the control system controls the electric stimulation coating to output a current of 0-15 mA. Raw materials of the electric heating coating include dendritic silver powder, flaky silver powder and a cross-linking agent, and raw materials of the electric stimulation coating include dendritic silver powder and a cross-linking agent.

This application claims the priority of Chinese Patent Application No. 202111339652.4, titled “CLOTH STRUCTURE HAVING ELECTRIC HEATING AND INSULATION AND ELECTRIC STIMULATION COATINGS AND PREPARATION METHOD THEREFOR”, filed on Nov. 12, 2021 with the China National Intellectual Property Administration, which is incorporated herein by reference in its entirety.

### FIELD

The present application relates to a cloth structure having an electric heating coating and an electric stimulation coating and a preparation method therefor.

### BACKGROUND

In order to keep up with the rising market of alleviation of soreness caused by various physical and mental syndromes as well as exercises, and meet the consumption demand of the coming aging society, along with the rapidly growing markets, a large number of targeted products emerge on the market especially in the photoelectric field, which are intended to meet the non-invasive requirements and take convenience and efficiency as the main purpose. In the treatments employ the principles of micro-current stimulation and thermal therapy by electricity, the application in relation to electric heating micro-current stimulation massage is most well-known, it has been gradually recognized by the mainstream medical profession, becoming a conventional relieving and therapeutic tool and widely used in self-therapy at home. Wearable devices for alleviating soreness are in accord with the development trend in the future, but the conventional products have the following shortcomings: (1) the traditional adhesive electrode patch is uncomfortable (with poor air permeability and obvious foreign body sensation) and inconvenient to use; (2) the main body is made of metal fibers, which has some problems, such as allergy is apt to happen, conductive wires are easily pulled out, corrosion and oxidization are apt to occur (leading to unstable electrical property), complicated tailoring procedures in the design and manufacturing process of smart wear, energy consumption and poor launderability, etc.

In the present invention, a soft washable woven cloth electrode is developed, and an electric heating coating and an electric stimulation coating are made in a post-processing manner (screen printing/transfer printing), and the coatings are combined with textiles in a simple wearing mode, to make the textiles have electric heating and electric stimulation functions, which improves the shortcomings of complicated wires of the conventional electric heating and electric stimulation equipment, so as to adapt to the rising market of alleviation of soreness caused by various physical and mental syndromes as well as exercises.

### SUMMARY

One of the technical problems to be solved by the present invention is to provide a cloth structure having an electric heating coating and an electric stimulation coating, which has electrical stimulation and electric heating functions, can perform electrical stimulation and hot compress on acupuncture points of the human body, to function to sooth and relax, relieve fatigue and so on, moreover, the cloth structure has advantages such as being light, thin, breathable, comfortable and washable.

The one of the technical problems is addressed as follows.

A cloth structure having an electric heating coating and an electric stimulation coating, which includes a substrate cloth, the electric heating coating, an insulation layer and the electric stimulation coating, where the substrate cloth has a first surface, the electric heating coating is attached to the first surface, the electric stimulation coating is attached to the electric heating coating via the insulation layer, the electric heating coating and the electric stimulation coating are electrically connected to a control system separately, the control system controls the electric heating coating to generate heat, and controls the electric stimulation coating to output a current ranging from 0 mA to 15 mA;
raw materials of the electric heating coating include dendritic silver powder and flaky silver powder, and a crosslinking agent, wherein a mass percentage concentration of the dendritic silver powder and the flake silver powder ranges from 35 wt % to 70 wt %; and
raw materials of the electric stimulation coating include dendritic silver powder and a crosslinking agent, wherein a mass percentage concentration of the dendritic silver powder ranges from 20 wt % to 60 wt %.

Further, a mass ratio of the dendritic silver powder to the flaky silver powder in the electric heating coating ranges from 0.05 to 1.

Further, the electric heating coating and the electric stimulation coating each has a thickness ranging from 0.05 mm to 2 mm.

Further, the dendritic silver powder and the flake silver powder each has a particle diameter ranging from 0.5 to 50 microns; and the crosslinking agent is at least one of polyurethane, epoxy resin, polymethyl methacrylate, polyvinyl chloride and polystyrene.

Further, an attachment manner of the electric heating coating and the electric stimulation coating is coating, screen printing or thermal transfer printing.

Further, the control system controls a temperature of the electric heating coating to be in a range from 40° C. to 55° C.; and the control system controls the electric stimulation coating to have a current output manner in a pulse mode and output a positive-negative symmetrical bidirectional rectangular pulse wave.

Another technical problem to be solved by the invention is to provide a method for preparing a cloth structure having an electric heating coating and an electric stimulation coating. The prepared product has electrical stimulation and electric heating functions, can perform electrical stimulation and hot compress on acupuncture points of the human body, to function to sooth and relax, relieve fatigue and so on, moreover, the cloth structure has advantages such as being light, thin, breathable, comfortable and washable.

The another technical problem is addressed as follows.

A method for preparing a cloth structure having an electric heating coating and an electric stimulation coating, including the following steps:
1) taking a certain amount of dendritic silver powder and flake silver powder with a particle diameter of 0.5 to 50 microns and adding the same into an appropriate amount of crosslinking agent solution, wherein a mass percentage concentration of the dendritic silver powder and the flake silver powder ranges from 35 wt % to 70 wt %; performing stirring treatment by at least one of an ultrasonic vibrator and a stirrer for 0.5 h to 1 h, to make the dendritic silver powder and the flake silver powder in the mixture be in a homogeneous state, to obtain uniformly dispersed electric heating paint; applying the electric heating paint on a substrate cloth through coating processing, screen printing or thermal transfer printing to form the electric heating coating; finally, placing the same in an oven for drying, curing and forming;
2) curing and forming an insulation layer on the electric heating coating;
3) taking a certain amount of dendritic silver powder with a particle diameter of 0.5 to 50 microns and adding the same into an appropriate amount of crosslinking agent solution, wherein a mass percentage concentration of the dendritic silver powder ranges from 20 wt % to 60 wt %; performing stirring treatment by at least one of the ultrasonic vibrator and the stirrer for 0.5 h to 1 h, to make the dendritic silver powder in the mixture be in a homogeneous state, to obtain uniformly dispersed electric stimulation paint; applying the electric stimulation paint on the insulation layer through coating processing, screen printing or thermal transfer printing to form the electric stimulation coating; finally, placing the same in an oven for drying, curing and forming; and
4) electrically connecting a control system to the electric heating coating and the electric stimulation coating separately, wherein the control system is configured to control the electric heating coating to generate heat and control the electric stimulation coating to output a current ranging from 0 mA to 15 mA, respectively;

Further, a mass ratio of the dendritic silver powder to the flaky silver powder in the electric heating coating ranges from 0.05 to 1.

Further, the electric heating coating and the electric stimulation coating each has a thickness ranging from 0.05 mm to 2 mm; and the crosslinking agent is at least one of polyurethane, epoxy resin, polymethyl methacrylate, polyvinyl chloride and polystyrene.

Further, the control system controls a temperature of the electric heating coating to be in a range from 40° C. to 55° C.; and the control system controls the electric stimulation coating to have a current output manner in a pulse mode and output a positive-negative symmetrical bidirectional rectangular pulse wave.

The present application has the following advantages.

The cloth structure of the present application has electrical stimulation and electric heating functions, which can perform electrical stimulation and hot compress on the acupuncture points of the human body, so as to promote the recovery of muscle tissue and enhance metabolism, thereby functioning to sooth and relax, relieve fatigue and alleviate soreness. The coatings of the present application can perform cross-link reaction with fibers, and can be applied to textiles by patterning energy-saving and environment-friendly manufacturing processes (coating, or screen printing, or transfer printing), the coating is light and thin, can be well fitted to skin and well adhered to textiles, has the advantages of energy saving (≤5 W), washable (≥50 times), and is durable, breathable and comfortable. Finally, the cloth structure of the present application can be made into a wearable structure, which can be worn or covered on the acupuncture points of human body to simultaneously or independently perform electrical stimulation and hot compress to acupuncture points of the human body percutaneously. The parts to be stimulated include the wrists, back and limbs, so as to integrally adjust the cardiovascular, skeletal and muscular systems of human body, and the present application has the advantages of being well-adapted, comfortable and the like, and can solve the shortcomings of complex manufacturing processes (conductive fibers) and being uncomfortable (conductive rubber) of conventional electric heating and low-frequency products.

### DETAILED DESCRIPTION OF THE EMBODIMENTS

Technical solutions of the present invention will be described clearly and completely in conjunction with specific embodiments hereinafter. All other embodiments obtained by those skilled in the art based on the embodiments in the present invention without any creative work fall in the scope of protection of the present invention. Those embodiments without indicating specific conditions were carried out according to the conventional conditions or the conditions suggested by the manufacturer. The reagents or instruments used without indicating manufacturer were all conventional products that could be commercially available.

The invention relates to a cloth structure having an electric heating coating and an electric stimulation coating, which includes a substrate cloth, the electric heating coating, an insulation layer and the electric stimulation coating, where the substrate cloth has a first surface, the electric heating coating is attached to the first surface, the electric stimulation coating is attached to the electric heating coating via the insulation layer, the electric heating coating and the electric stimulation coating are electrically connected to a control system separately, the control system controls the electric heating coating to generate heat, and the control system controls the electric stimulation coating to output a current ranging from 0 mA to 15 mA;
the raw materials of the electric heating coating include dendritic silver powder and flaky silver powder, and a crosslinking agent, where a mass percentage concentration of the dendritic silver powder and flake silver powder ranges from 35 wt % to 70 wt %; and
the raw materials of the electric stimulation coating include dendritic silver powder and a crosslinking agent, wherein a mass percentage concentration of the dendritic silver powder ranges from 20 wt % to 60 wt %.

A mass ratio of the dendritic silver powder to the flaky silver powder in the electric heating coating ranges from 0.05 to 1.

The electric heating coating and the electric stimulation coating each has a thickness ranging from 0.05 mm to 2 mm.

The dendritic silver powder and the flake silver powder each has a particle diameter ranging from 0.5 to 50 microns; and the crosslinking agent is at least one of polyurethane, epoxy resin, polymethyl methacrylate, polyvinyl chloride and polystyrene.

An attachment manner of the electric heating coating and the electric stimulation coating is coating, screen printing or thermal transfer printing.

The control system controls a temperature of the electric heating coating to be in a range from 40° C. to 55° C.; and the control system controls the electric stimulation coating to have a current output manner in a pulse mode and output a positive-negative symmetrical bidirectional rectangular pulse wave.

The present application also relates to a method for preparing the cloth structure having the electric heating coating and the electric stimulation coating, which includes the following steps:
1) taking a certain amount of dendritic silver powder and flake silver powder with a particle diameter of 0.5 to 50 microns and adding the same into an appropriate amount of crosslinking agent solution, where a mass percentage concentration of the dendritic silver powder and the flake silver powder ranges from 35 wt % to 70 wt %; performing stirring treatment by at least one of an ultrasonic vibrator and a stirrer for 0.5 h to 1 h, to make the dendritic silver powder and the flake silver powder in the mixture be in a homogeneous state, to obtain uniformly dispersed electric heating paint; applying the electric heating paint on a substrate cloth through coating processing, screen printing or thermal transfer printing to form the electric heating coating; finally, placing the same in an oven for drying, curing and forming;
2) curing and forming an insulation layer on the electric heating coating;
3) taking a certain amount of dendritic silver powder with a particle diameter of 0.5 to 50 microns and adding the same into an appropriate amount of crosslinking agent solution, where a mass percentage concentration of the dendritic silver powder ranges from 20 wt % to 60 wt %; performing stirring treatment by at least one of the ultrasonic vibrator and the stirrer for 0.5 h to 1 h, to make the dendritic silver powder in the mixture be in a homogeneous state, to obtain uniformly dispersed electric stimulation paint; applying the electric stimulation paint on the insulation layer through coating processing, screen printing or thermal transfer printing to form the electric stimulation coating; finally, placing the same in the oven for drying, curing and forming; and
4) electrically connecting a control system to the electric heating coating and the electric stimulation coating separately, where the control system is configured to control the electric heating coating to generate heat and control the electric stimulation coating to output a current ranging from 0 mA to 15 mA, respectively.

The control system can apply a voltage of 5V to the electric heating coating, to make the temperature the electric heating coating be in a range from 40° C. to 55° C., and can adjust the temperature and service time at the same time. The current output by the electric stimulation coating ranges from 0 to 15 mA, and is adjustable continuously in the whole process. The current is output in a manner of pulses, which is embodied as a positive-negative symmetrical bidirectional rectangular pulse wave. A variety of waveforms may be output, including dense-sparse constant amplitude wave, dense constant amplitude wave and amplitude modulation wave (2 Hz to 100 Hz).

The insulation layer is a polymer compound layer with a thickness ranging from 0.05 mm to 2 mm, and its specific components may be at least one of polyurethane, epoxy resin, polymethyl methacrylate, polyvinyl chloride and polystyrene.

The cloth structure of the present application has electrical stimulation and electric heating functions, which can perform electrical stimulation and hot compress on the acupuncture points of the human body, so as to promote the recovery of muscle tissue and enhance metabolism, thereby functioning to sooth and relax, relieve fatigue and alleviate soreness. The coatings of the present application can perform cross-link reaction with fibers, and can be applied to textiles by patterning energy-saving and environment-friendly manufacturing processes (coating, or screen printing, or transfer printing), the coating is light and thin, can be well fitted to skin and well adhered to textiles, has the advantages of energy saving (≤5 W), washable (≥50 times), and is durable, breathable and comfortable. Finally, the cloth structure of the present application can be made into a wearable structure, which can be worn or covered on the acupuncture points of human body to simultaneously or independently perform electrical stimulation and hot compress to acupuncture points of the human body percutaneously. The parts to be stimulated include the wrists, back and limbs, so as to integrally adjust the cardiovascular, skeletal and muscular systems of human body, and the present application has the advantages of being well-adapted, comfortable and the like, and can solve the shortcomings of complex manufacturing processes (conductive fibers) and being uncomfortable (conductive rubber) of conventional electric heating and low-frequency products.

### First Embodiment

A kneepad having an electric heating coating and an electric stimulation coating was provided according to this embodiment, including a substrate cloth, the electric heating coating, an insulation layer and the electric stimulation coating, where the substrate cloth had a first surface, the electric heating coating was attached to the first surface, the electric stimulation coating was attached to the electric heating coating via the insulation layer, the electric heating coating and the electric stimulation coating were electrically connected to a control system separately, the control system controlled the electric heating coating to generate heat. The electric stimulation coating had a thickness of 0.1 mm, and included dendritic silver powder and polyurethane, where the dendritic silver powder accounted for 50% of the electric stimulation coating in mass. The electric heating coating had a thickness of 0.1 mm, and included dendritic silver powder, flake silver powder and polyurethane, where the total amount of the dendritic silver powder and the flake silver powder accounted for 50% of the electric heating coating in mass, and a mass ratio of the dendritic silver powder to the flake silver powder was 0.2:1. The dendritic silver powder and the flake silver powder each had a particle diameter of 20 microns. The insulation layer was made of epoxy resin and had a thickness of 0.1 mm.

The kneepad was heated with a voltage of DC 5V and a current of 0.7 A.

Kneepad area: 8 cm*14 cm, effective thickness of electric heating coating: 0.1 mm, effective area of electric heating coating: 5 cm*10 cm, target temperature: 45° C.; effective thickness of electric stimulation coating: 0.1 mm, effective area of electric stimulation coating: 5 cm*10 cm, and the output current for electric stimulation was of 5 mA, which was a dense constant amplitude wave having a frequency of 50 Hz and a wave width of 100 ms.

The temperature test is shown as follows:

| Time (s) | 0 | 10 | 20 | 30 | 40 | 50 | 60 |
|---|---|---|---|---|---|---|---|
| Temperature (° C.) | 30 | 32.5 | 35.7 | 38.7 | 41.1 | 43.6 | 45.3 |

Although the specific embodiments of the present application have been described above, those skilled in the art should understand that the specific embodiments are only illustrative, and are not used to limit the scope of the present application. Equivalent modifications and changes made by those skilled in the art in accordance with the spirit of the present application should be covered by the scope of the claims of the present application.

1. A cloth structure having an electric heating coating and an electric stimulation coating, comprising a substrate cloth, the electric heating coating, an insulation layer and the electric stimulation coating, wherein
the substrate cloth has a first surface, the electric heating coating is attached to the first surface, the electric stimulation coating is attached to the electric heating coating via the insulation layer;
the electric heating coating and the electric stimulation coating are electrically connected to a control system separately, the control system is configured to control the electric heating coating to generate heat, and control the electric stimulation coating to output a current ranging from 0 mA to 15 mA; wherein
raw materials of the electric heating coating comprise dendritic silver powder and flaky silver powder, and a crosslinking agent, wherein a mass percentage concentration of the dendritic silver powder and the flake silver powder ranges from 35 wt % to 70 wt %; and
raw materials of the electric stimulation coating comprise dendritic silver powder and a crosslinking agent, wherein a mass percentage concentration of the dendritic silver powder ranges from 20 wt % to 60 wt %.

2. The cloth structure having the electric heating coating and the electric stimulation coating according to claim 1, wherein a mass ratio of the dendritic silver powder to the flaky silver powder in the electric heating coating ranges from 0.05 to 1.

3. The cloth structure having the electric heating coating and the electric stimulation coating according to claim 1, wherein the electric heating coating and the electric stimulation coating each has a thickness ranging from 0.05 mm to 2 mm.

4. The cloth structure having the electric heating coating and the electric stimulation coating according to claim 1, wherein the dendritic silver powder and the flake silver powder each has a particle diameter ranging from 0.5 to 50 microns; and the crosslinking agent is at least one of polyurethane, epoxy resin, polymethyl methacrylate, polyvinyl chloride and polystyrene.

5. The cloth structure having the electric heating coating and the electric stimulation coating according to claim 1, wherein an attachment manner of the electric heating coating and the electric stimulation coating is coating, screen printing or thermal transfer printing.

6. The cloth structure having the electric heating coating and the electric stimulation coating according to claim 1, wherein the control system is configured to control a temperature of the electric heating coating to be in a range from 40° C. to 55° C.; and the control system is configured to control the electric stimulation coating to have a current output manner in a pulse mode and output a positive-negative symmetrical bidirectional rectangular pulse wave.

7. A method for preparing a cloth structure having an electric heating coating and an electric stimulation coating, comprising:
1) taking a certain amount of dendritic silver powder and flake silver powder with a particle diameter of 0.5 to 50 microns and adding the dendritic silver powder and the flake silver powder into an appropriate amount of crosslinking agent solution, wherein a mass percentage concentration of the dendritic silver powder and the flake silver powder ranges from 35 wt % to 70 wt %; performing stirring treatment by at least one of an ultrasonic vibrator and a stirrer for 0.5 h to 1 h, to make the dendritic silver powder and the flake silver powder in the mixture be in a homogeneous state, to obtain uniformly dispersed electric heating paint; applying the electric heating paint on a substrate cloth through coating processing, screen printing or thermal transfer printing to form the electric heating coating; finally, placing the same in an oven for drying, curing and forming;
2) curing and forming an insulation layer on the electric heating coating;
3) taking a certain amount of dendritic silver powder with a particle diameter of 0.5 to 50 microns and adding the dendritic silver powder into an appropriate amount of crosslinking agent solution, wherein a mass percentage concentration of the dendritic silver powder ranges from 20 wt % to 60 wt %; performing stirring treatment by at least one of the ultrasonic vibrator and the stirrer for 0.5 h to 1 h, to make the dendritic silver powder in the mixture be in a homogeneous state, to obtain uniformly dispersed electric stimulation paint; applying the electric stimulation paint on the insulation layer through coating processing, screen printing or thermal transfer printing to form the electric stimulation coating; finally, placing the same in the oven for drying, curing and forming; and
4) electrically connecting a control system to the electric heating coating and the electric stimulation coating separately, wherein the control system is configured to control the electric heating coating to generate heat and control the electric stimulation coating to output a current ranging from 0 mA to 15 mA, respectively.

8. The method for preparing the cloth structure having the electric heating coating and the electric stimulation coating according to claim 7, wherein a mass ratio of the dendritic silver powder to the flaky silver powder in the electric heating coating ranges from 0.05 to 1.

9. The method for preparing the cloth structure having the electric heating coating and the electric stimulation coating according to claim 7, wherein the electric heating coating and the electric stimulation coating each has a thickness ranging from 0.05 mm to 2 mm; and the crosslinking agent is at least one of polyurethane, epoxy resin, polymethyl methacrylate, polyvinyl chloride and polystyrene.

10. The method for preparing the cloth structure having the electric heating coating and the electric stimulation coating according to claim 7, wherein the control system controls a temperature of the electric heating coating be in a range from 40° C. to 55° C.; and the control system controls the electric stimulation coating to have a current output manner in a pulse mode and output a positive-negative symmetrical bidirectional rectangular pulse wave.
